(19) United States
(12) Patent Application Publication
(10) Pub. No.: US 2010/0114067 A1
(43) Pub. Date: May 6, 2010

(54) Multi-Chamber Mixing System

(75) Inventors: Hai H. Trieu, Cordova, TN (US); Sean M. Haddock, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/262,905

(22) Filed: Oct. 31, 2008

(51) Int. Cl. A61M 5/31

(52) U.S. Cl. 604/518; 604/91

(57) ABSTRACT

Mixing systems for storing and mixing multiple components of various medical compositions are provided. Generally, the instant mixing systems comprise a barrel, a plunger slidably disposed inside a barrel, and a pushing rod slidably disposed inside the plunger. The instant mixing system may further comprise multiple chambers for storing individual components or mixtures of components, with at least one chamber disposed in the barrel and at least one chamber disposed inside the plunger.

### FIELD OF THE INVENTION

This invention is related to mixing systems for storing and mixing individual components of medical compositions and administering these compositions to patients. More particularly, this invention is related to mixing systems with multiple, isolated chambers.

### BACKGROUND

Various medical compositions, such as bone fillers, bone cements, and dental and medical adhesives, often require combining together several components in liquid or solid form. Typically, the individual components of such compositions are separated from each other during storage and are combined immediately prior to their administration to a patient.

To be successful a system for storing and mixing individual components for medical compositions needs to meet a number of objectives. First, it needs to be capable of storing components in both liquid and solid form and to ensure that these components are completely isolated from each other during storage and transportation of the mixing system. Second, a mixing system needs to enable mixing of the individual components that is rapid and thorough and requires minimum effort by the user. Third, although many separate components may be involved, a mixing system needs to be designed in such a way as to minimize its size in order to facilitate convenient handling, transportation and storage of the mixing system.

Although many different mixing systems are currently available, none of them meets all of these objectives. Accordingly, there is still a need in the art for an improved mixing system for mixing medical compositions and administering them to a patient.

### SUMMARY

Mixing systems for storing and mixing components for multi-component medical compositions are provided.

In one embodiment, a multiple-component mixing system may comprise a first barrel comprising at least one first chamber, a plunger slidably disposed inside the barrel, at least one second chamber disposed inside the plunger and fluidly isolated from the at least one first chamber; and a pushing rod disposed inside the plunger proximally of the at least one second chamber.

In another embodiment, a multiple-component mixing system may comprise a barrel comprising at least one first chamber and at least one second chamber fluidly isolated from each other, a plunger slidably disposed inside the barrel, at least one third chamber disposed inside the plunger and fluidly isolated from the at least one first chamber and the at least one second chamber, and a pushing rod disposed inside the plunger. The second chamber may be disposed between the plunger and inner walls of the barrel.

In yet another embodiment, a multiple-component mixing system may comprise a first barrel and a second barrel fluidly connected to a common outlet. Furthermore, at least one of the barrels may comprise at least one first chamber, a plunger slidably disposed inside at least one of the barrels, at least one second chamber disposed inside the plunger and fluidly isolated from the at least one first chamber, and a pushing rod disposed inside the plunger proximally of the at least one second chamber. The barrel with multiple chambers may further include at least one third chamber formed between the interior wall of the barrel and the outer wall of the plunger.

In yet another embodiment, the invention provides a method of mixing multiple-components in the same device in which they were stored and delivering the mixed multi-component composition to a target site.

In all aspects, the pushing rod is preferably adapted to push a component from the chamber inside the plunger into a chamber in the barrel. Accordingly, each chamber in the instant mixing system may be used for storing individual components in various forms such as liquid, solid, or powder.

### BRIEF DESCRIPTION OF THE FIGURES

FIG. 1 presents one embodiment of the instant multiple component mixing system.

FIGS. 2-5 present use of one embodiment of the mixing system of FIG. 1.

FIGS. 5-7 present use of another embodiment of the mixing system of FIG. 1.

FIGS. 8-9 present one embodiment of a closable mixing channel of the mixing system of FIGS. 5-7.

FIG. 10 presents another embodiment of the instant multiple component mixing system.

FIGS. 11-13 present use of the mixing system of FIG. 10.

FIG. 14 presents yet another embodiment of the instant multiple component mixing system.

### DETAILED DESCRIPTION

Mixing systems for storing and mixing multiple components of various medical compositions are provided. In particular, such systems may be used for administering bone cements in orthopedic procedures, such as for anchoring artificial joints, or in spine related procedures, such as the treatment of vertebral compression fractures and traumatic reconstructions.

Generally, the instant mixing systems comprise a barrel, a plunger slidably disposed inside a barrel, and a pushing rod slidably disposed inside the plunger. The instant mixing system may further comprise multiple chambers for storing individual components or mixtures of components, with at least one chamber disposed in the barrel and at least one chamber disposed inside the plunger. The total volume of the instant mixing system could feasibly be between about 5 cc to about 50 cc. The volume of individual chambers within the instant mixing system may range between about 1 cc to about 20 cc.

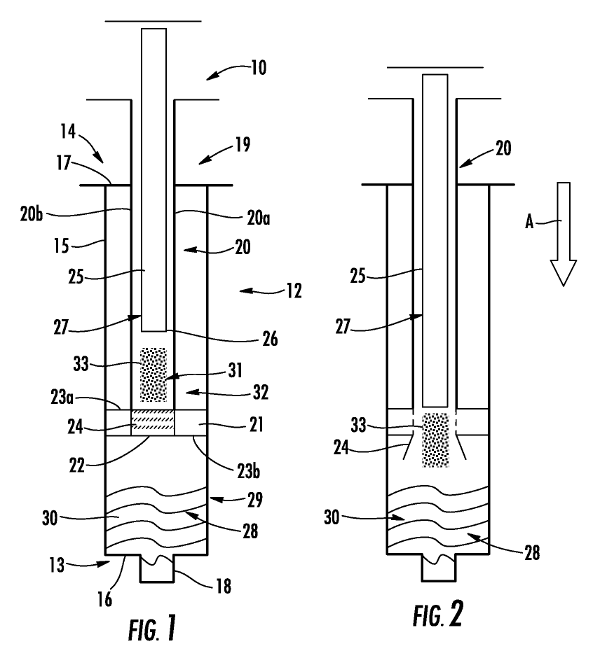

FIG. 1 presents one embodiment of the instant multiple-component mixing, dispensing and/or injecting system. Such mixing and dispensing system 10 comprises a barrel 12 having a distal end 13, a proximal end 14, side walls 15, distal walls 16, and, optionally, proximal walls 17. A nozzle 18, disposed at the distal end 13 of the barrel 12, facilitates attachment to the barrel 12 of a needle, a catheter or any other devices through which the contents of the mixing system may be administered to a patient.

A plunger 19 is slidably disposed inside the barrel 12. The plunger 19 comprises a generally cylindrical hollow plunger shaft 20 with side walls 20a, 20b and a plunger cap 21, preferably, sized to form a seal with the interior walls of the barrel 12. In some embodiments, the outer diameter of plunger shaft 20 may be approximately the same as the inner diameter of the barrel 12 so a seal is formed between the plunger 19 and the barrel 12 to prevent components inside the barrel from spilling out, and thus the proximal walls of the barrel 12 may be omitted. Alternatively, the outer diameter of the plunger shaft 20 may be smaller than the inner diameter of the barrel 12 to define an additional chamber, and thus the proximal walls may be needed, unless the additional chamber is sealed by other means. The plunger cap 21 comprises a central opening 22, a proximal side 23a and a distal side 23b. Initially, the central opening 22 may be by sealed by means of a frangible seal 24. As used herein, the term frangible seal means a seal that has sufficient integrity to avoid breakage during storage and normal handling, but that can be intentionally separated or ruptured when desired without undue effort and without creating particulates that may be harmful to a patient. Suitable materials for the frangible seal may include, but are not limited to, rubber, silicone, polyurethane, or Teflon.

A pushing rod 25 having a distal end 26 may be disposed within the plunger 19. The pushing rod 25 is preferably sized so at least the distal region 27 of the pushing rod 25 forms a tight seal with the interior walls of the plunger and fits tightly into the central opening 22 in the plunger cap 21. The pushing rod 25 is adapted to push the material from the chamber inside the plunger. For example, the pushing rod may comprise at least one section that is sized to form a seal between the pushing rod and the plunger distally of the chamber inside the plunger.

The multiple-component mixing system 10 may comprise multiple chambers for storing individual components. At least one chamber, such as a first chamber 28, may be situated in the distal region 29 of the barrel 12. Such first chamber may be defined by side walls 15 and distal walls 16 of the barrel 12 and the distal side 23b of the plunger cap 21. The first chamber 28 may contain at least one component, such as a first component 30 either in solid, powder, or liquid form. The first component may comprise a single component or a mixture of components. In addition, at least one chamber, such as a second chamber 31, may be located in the distal region 32 of the plunger 19. Such second chamber may be defined by the interior walls of the plunger 19, the distal end of the pushing rod 25, and the proximal side of the plunger. The second chamber 31 may hold a single component or a mixture of components, such as a second component 33, either in solid, powder, or liquid form.

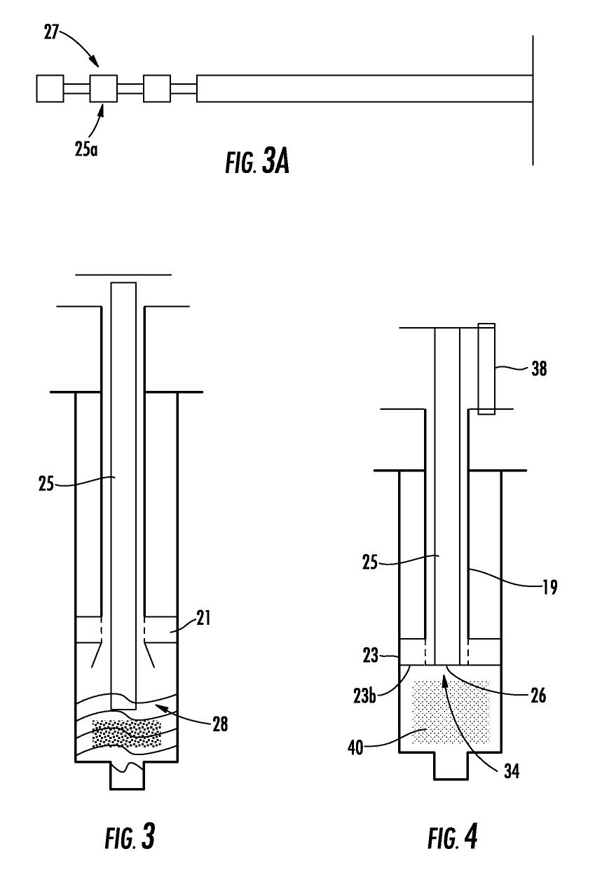

FIG. 1 shows an embodiment of the instant multi-component mixing system in its initial state, such as storage or transportation. In the initial state, the first chamber 28 and the second chamber 31 are isolated from each other and are filled with the first component 30 and the second component 33, respectively. As shown in FIG. 2, to add the second component 32 to the first chamber 28, the pushing rod 25 may be moved in the distal direction, which is indicated by arrow A. Moving the pushing rod 25 in the distal direction generates a force in the distal direction on the second component 33 causing the frangible seal 24 to break and to release the second component 33 into the first chamber 28. Accordingly, as noted above, the distal region 27 of the pushing rod 25 preferably forms a tight seal with the interior walls of the plunger shaft 20 to prevent the second component 33 from traveling in the proximal direction into the space between the plunger and the pushing rod.

Once the second component 33 is added to the first chamber 28, the first component 30 and the second component 33 may be mixed by any known technique. In one embodiment, the first and second component may be mixed inside the first chamber 28. For example, as shown in FIG. 3, the pushing rod 25 may extend past the plunger cap 21 into the first chamber 28, and the first and second components may be mixed by oscillating or spinning the pushing rod 25. To aid mixing of the components, the pushing rod 25 may comprise a mixing section 25a disposed in the distal region 27 of the pushing rod 25, such as, for example, sections with varying diameters, as shown in FIG. 3a, or similar. Alternatively, the first component may be mixed with the second component by shaking or vibrating the mixing system.

Referring to FIG. 4, once the components are sufficiently mixed to result in a uniform mixture 40, the pushing rod 25 may be withdrawn back into the plunger 19, preferably aligning the distal end 26 of the pushing rod 25 with the distal side 23b of the plunger cap 23 to form a continuous surface 34 on the distal side 23b of the plunger cap 23. Accordingly, it is desirable that the distal region of the pushing rod 25 is sized to form a tight seal with the central opening to prevent the component mixture from flowing into the plunger. In some embodiments, the pushing rod 25 may be locked in that position by a lock 38.

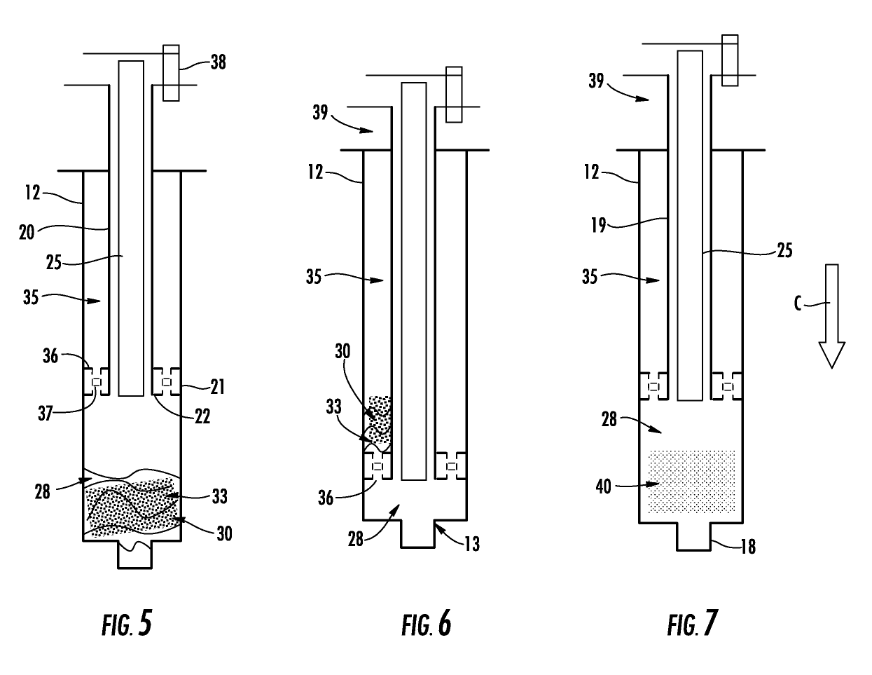

In another embodiment, the mixing system 10 may include at least one mixing chamber, such as the first mixing chamber 35, for mixing individual components. In such embodiment, the outer diameter of the plunger shaft 20 is smaller than the inner diameter of the barrel 12, so the first mixing chamber 35 is formed between the inner walls of the barrel 12 and the outer wall of the plunger shaft 20. Additionally, in such embodiments, the plunger cap 21 may include at least one mixing channel, such as a first mixing channel 36, to allow the components to flow from the first chamber 28 to the mixing chamber 35. Preferably, the first mixing channel 36 includes a mixing element 37 to achieve better mixing of the components. Suitable examples of the mixing element include, but are not limited, to a static mixer with a range of mixing elements, sizes, and shapes or a rotating or exposed impeller. Operation of this embodiment of the mixing system is shown in FIGS. 5-7.

Referring to FIG. 5, after the second component 33 is added to the first component 30, the pushing rod 25 may be inserted into the central opening 22 of the plunger cap 21 to plug the central opening 22, as described above. In some embodiment, the pushing rod may be locked in this position by the lock 38.

Next, the plunger 19 and the pushing rod assembly 39 is moved in the proximal direction toward the distal end 13 of the barrel 12, as shown in FIG. 6. The plunger and the pushing rod assembly 39 may be moved manually, with a ratchet, or a syringe pump. Because the nozzle 18 is preferably sealed during mixing, moving the plunger and pushing rod assembly 39 in the distal direction forces the first component 30 and the second component 33 to flow from the first chamber 28 through the mixing channel 36 into the first mixing chamber 35. Once the components are transferred from the first chamber 28 into the first mixing chamber 35, the plunger and the mixing rod assembly 39 is moved back in the proximal direction. This forces the mixture of components 40 to flow back from the first mixing chamber 36 into the first chamber 28, as shown in FIG. 7. The steps shown in FIGS. 5 though 7 may be repeated to further mix the components as necessary or desired.

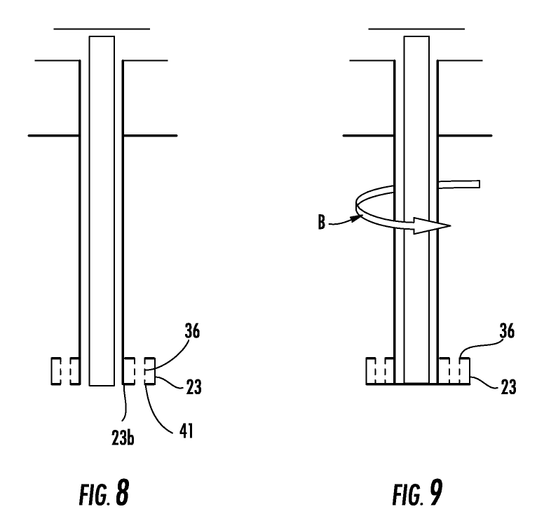

To extrude the mixture of components 40, the first mixing channel 36 is preferably closed. For example, referring to FIG. 8, the distal side 23b of the plunger cap 21 may be rotatable relative to the proximal side 23a and may include at least one opening 41. To open the first mixing channel, the at least one opening 41 in the distal side 23b of the plunger cap 21 may be aligned with the first mixing channel 36. Referring to FIG. 9, to close the first mixing channel 36, the distal side 23b may be rotated by, for example, rotating the plunger shaft 20 as represented by arrow B, so the at least one opening 41 in the distal side 23b of the plunger cap 21 no longer aligns with the first mixing channel 36, thus closing the first mixing channel 36. However, the channels may not need to be closed if, when the mixture is pushed in the distal direction, it is more likely to flow out from the mixing system than into the mixing chamber.

Referring back to FIG. 7, once the first component and the second component are sufficiently mixed to form the mixture 40, the plunger and the pushing rod assembly 39 may be moved in the distal direction, as shown by arrow C, to force the mixture of components 40 out of the barrel 12 through the nozzle 18 and into a syringe, a catheter, or any other device for administering the mixture from the mixing system to a patient. In some embodiments, the nozzle may be sealed with a frangible seal. Such seal is preferably designed to withstand the pressure generated when the components are being mixed and to break due to pressure generated when the mixture is being extruded out of the barrel. In other embodiments, the nozzle may be closed by mechanical means such as a lid or a cap.

In another aspect, the instant mixing system may include more than two chambers and, thus, may be used to prepare mixtures requiring more than two components. In particular, the instant mixing systems may be used to prepare epoxy-like materials that have more than one liquid part, and may require the separate storage of a powdered or dry initiator for polymerization.

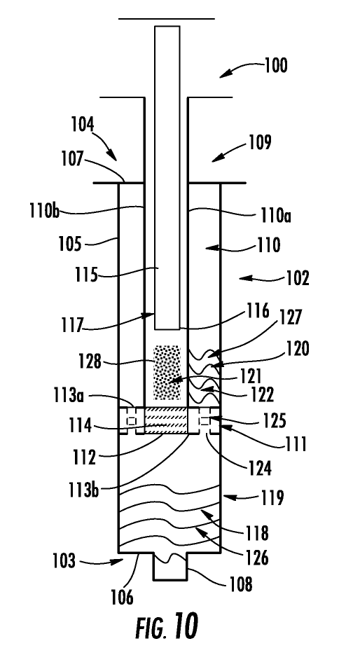

One embodiment of the instant mixing system comprising more than two chambers is presented in FIG. 10. Such system is similar to the embodiments described above, except that in addition to the at least one chamber in the distal region of the barrel and at least one chamber in the distal region of the plunger, it may also include at least one additional chamber located between the walls of the barrel and the plunger. For example, the additional chamber may be defined by the inner wall of the barrel, the proximal wall of the barrel, the outer wall of the plunger, and the proximal side of the plunger cap.

Referring to FIG. 10, the multiple-component mixing system 100 comprises a barrel 102 having a distal end 103, a proximal end 104, side walls 105, distal walls 106, and optionally proximal walls 107. A nozzle 108, disposed at the distal end 103 of the barrel 102, facilitates attachment to the barrel 102 of a needle, a catheter or other devices through which the contents of the mixing system may be administered.

A plunger 109 is slidably disposed inside the barrel 102. The plunger 109 comprises a generally cylindrical hollow plunger shaft 110 with side walls 110a, 110b and a plunger cap 111, preferably, sized to form a seal with the interior walls of the barrel 102. The outer diameter of at least one section of the plunger shaft 110 is smaller than the inner diameter of the barrel 102 to accommodate a chamber, and thus the proximal walls 107 of the barrel 102 seal the chamber at its proximal end, unless the chamber is sealed by other means. The plunger cap 111 comprises a central opening 112, a proximal side 113a and a distal side 113b. Initially, the central opening 112 may be by sealed by means of a frangible seal 114, as defined above.

A pushing rod 115 having a distal end 116 may be disposed within the plunger 119. The distal end of the pushing rod 115 preferably fits tightly into the central opening 112 in the plunger cap 111 to form a seal. The pushing rod 115 is adapted to push the material from the chamber inside the plunger. For example, the pushing rod may comprise at least one section that is sized to form a seal between the pushing rod and the plunger distally of the chamber inside the plunger.

The multiple-component mixing system 100 may comprise multiple chambers for storing individual components. The chambers are isolated from each other during storage and transportation of the mixing systems. Each chamber may store a single component or a mixture of components in either in solid, powder, or liquid form. At least one chamber, such as a first chamber 118, may be situated in the distal region 119 of the barrel 102 defined by side walls 105 and distal walls 106 of the barrel 102 and the distal side 113b of the plunger cap 111. At least one additional chamber, such as a second chamber 120, may be located proximally of the first chamber 118 between the inner walls of the barrel 102 and outer wall of the plunger shaft 110. Yet another chamber, such as a third chamber 121, may be located in the distal region 122 of the plunger 109. In the preferred embodiments, the third chamber may be defined by the interior walls of the plunger 109, the proximal side of the plunger cap 113a and the distal end of the pushing rod 116. Alternatively, the pushing rod may have a section with the smaller diameter than the inner diameter of the plunger, and the third chamber may be disposed between the pushing rod and the wall of the plunger along such section. However, it is desirable that the diameter of the pushing rod proximally of the third chamber is selected so the pushing rod and the interior walls of the plunger form a tight seal proximally of the third chamber.

The plunger cap 113 may include at least one channel 124 between the first chamber 118 and the second chamber 120. The at least one channel 124 may be open and closed as described above in regard to the mixing chambers and shown in FIGS. 8 and 9. Alternatively, the at least one channel may include check valves that permit flow from the second chamber to the first chamber but not the other way. To achieve more thorough mixing, the at least one mixing channel 124 may comprise a mixing element 125.

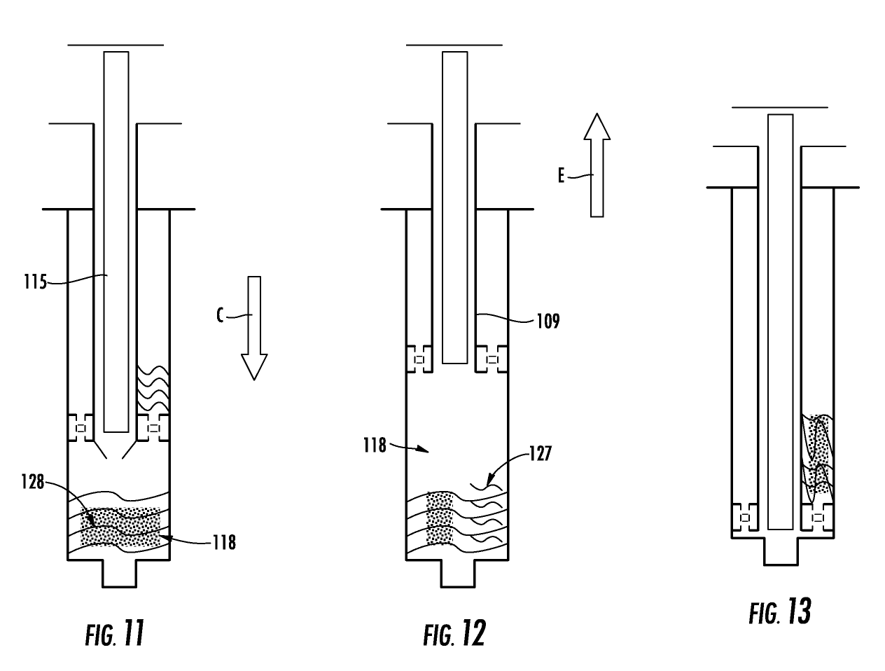

FIG. 10 shows the multi-component mixing system in its initial state, such as storage or transportation. In this state, the first chamber 118, the second chamber 120, and the third chamber 121 are isolated from each other and are filled with the first component 126, the second component 127, and the third component 128, respectively. Referring to FIG. 11, to add the third component 128 to the first chamber 118, the pushing rod 115 may be moved in the distal direction, as indicated by arrow D. To add the second component 127 to the first chamber 118, the plunger 109 may be moved in the proximal direction, as shown in FIG. 12, or in the distal direction. Alternatively, in embodiments without check valves, the components in the first chamber may be mixed with components in the second chamber by moving the plunger in the distal direction, as shown in FIG. 13.

Next, the components are mixed so the mixture may be administered to a patient. In some embodiments, all components may be collected in the same chamber and then mixed in a single step. In other embodiments, select components may be mixed first with the other components added to the mixture at a later time. The mixing may be achieved in a variety of ways depending on the embodiment of the mixing system, the sequence of mixing and so forth. For example, if the individual components need to be mixed in the first chamber, such as if the first and second component need to be pre-mixed prior to addition of the third component or in the embodiments with the check valves, the components may be mixed with the assistance of the pushing rod as described above. Alternatively, in the embodiments without the check valves, the third chamber may be used as the mixing chamber as described above and is shown in FIGS. 5-7.

Once all components are sufficiently mixed, the pushing rod is withdrawn into the plunger, as described above, and the plunger and the pushing rod assembly may be moved in the distal direction to force the mixture of component out of the barrel through the nozzle to be administered to a patient. In the embodiments without check valves, the channels are preferably closed as described above.

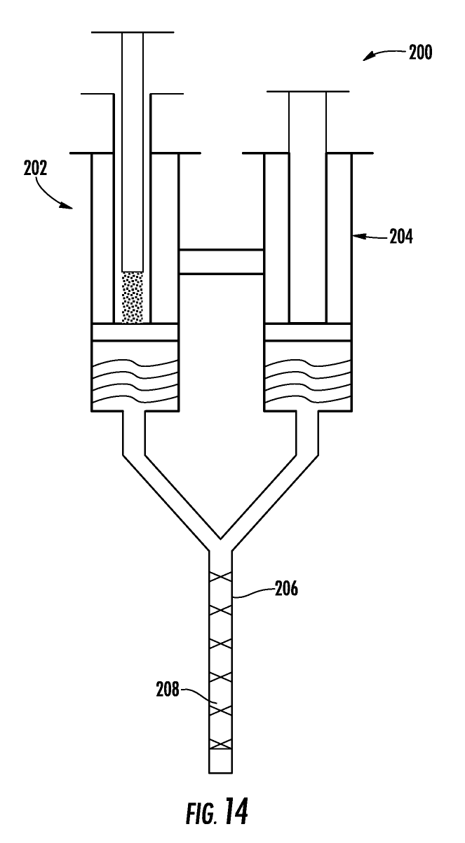

Another embodiment of the instant mixing system comprising more than two chambers is presented in FIG. 14. In this embodiment, the mixing system 200 comprises multiple barrels, such as the first barrel 202 and the second barrel 204. At least one of the barrels may comprise two chambers or more than two chambers, as described above. Although FIG. 14 shows the multiple-component mixing systems comprising of two barrels with only one of the barrels comprising two chambers, it will be understood that in various embodiments more than two barrels may be employed and any number of the barrels may comprise two chambers or more than two chambers.

The first barrel 202 and the second barrel 204 are connected to the common outlet 206. The common outlet 206 may include a static mixer 208 to achieve better mixing of components or mixtures of components from various barrels. In practice, if a barrel holds multiple components, these components may be mixed within that barrel by any method described above and the resulting mixture may then be mixed with contents of other barrels. In some embodiments including more than two barrels, all barrels may be connected to the same common outlet so their contents may be mixed at the same time. In other embodiments, the contents of select barrels may be mixed first and the contents of other barrels may be added at a later time.

The instant mixing systems allow mixing multiple components together within a single device. By way of non-limiting example, some in situ curing formulations comprising at least two liquid components may have stability issues, which may cause the individual components to separate when administered to a patient. If the individual components separate, the formulation may only partially cure or not cure at all, and additional amounts of the formulation may have to be administered to the patient, thus increasing the cost and duration of the medical procedure. Hence, there may be a need to mix a stabilizing component, usually in the form of powder, with one liquid component prior to mixing the liquid components together.

Accordingly, the first liquid component and the second liquid component may be stored in the first chamber and second chamber, respectively, of the instant mixing system. The stabilizing component in the form of powder may be stored in the third chamber inside the plunger. To prepare the formulation, the stabilizing component may be added to the first chamber and thoroughly mixed with the first liquid component, as described above. Then, the mixture of the first liquid component and the stabilizing component may then be mixed with the second liquid component, as described above, to produce a stable liquid formulation.

Note that the specifics embodiments are described in an exemplary manner and are not intended to limit the invention. In particular, mixing systems manufactured of any acceptable material are contemplated to be within the scope of the invention, as are mixing systems having varying design configurations and numbers of chambers. The scope of the invention is therefore defined in the claims which follow.

What is claimed is:

1. A multiple-component mixing system comprising:
a first barrel comprising at least one first chamber;
a plunger slidably disposed inside the first barrel;
at least one second chamber disposed inside the plunger and fluidly isolated from the at least one first chamber; and
a pushing rod disposed inside the plunger proximally of the at least one second chamber.

2. The mixing system of claim 1, wherein the at least one second chamber is isolated from the at least one first chamber by a frangible seal.

3. The mixing system of claim 1, wherein the pushing rod is adapted to push a component from the at least one second chamber into the at least one first chamber.

4. The mixing system of claim 1, wherein the pushing rod comprises a mixing section.

5. The mixing system of claim 1, wherein the plunger comprises a plunger shaft and a plunger cap.

6. The mixing system of claim 5 further comprising at least one mixing chamber formed between the interior wall of the barrel and the outer wall of the plunger shaft.

7. The mixing system of claim 5, wherein the plunger cap comprises at least one mixing channel.

8. The mixing system of claim 7, wherein the mixing channel comprises a mixing element.

9. The mixing system of claim 1, further comprising a second barrel fluidly isolated from the first barrel and comprising a third chamber wherein the first barrel and the second barrel are fluidly connected to a common channel.

10. The mixing system of claim 9, further comprising a static mixer disposed inside the common channel.

11. A multiple-component mixing system comprising:
a barrel comprising at least one first chamber and at least one second chamber fluidly isolated from each other;
a plunger slidably disposed inside the barrel;
at least one third chamber disposed inside the plunger and fluidly isolated from the at least one first chamber and the at least one second chamber; and
a pushing rod disposed inside the plunger.

12. The mixing system of claim 11, wherein the plunger is disposed proximally of the at least one third chamber.

13. The mixing system of claim 11, wherein the second chamber is disposed between the plunger and inner walls of the barrel.

14. The mixing system of claim 13, wherein the plunger comprises closable channels.

15. The mixing system of claim 11, wherein the pushing rod is adapted to push a component from the at least one third chamber into the at least one first chamber.

16. A multiple-component mixing system comprising:
a first barrel and a second barrel, wherein at least one of the first barrel or second barrel comprises:
at least one first chamber;
a plunger slidably disposed inside the barrel;
at least one second chamber disposed inside the plunger and fluidly isolated from the at least one first chamber; and
a pushing rod disposed inside the plunger proximally of the at least one second chamber.

17. The mixing system of claim 16 further comprising at least one third chamber formed between the interior wall of the barrel and the outer wall of the plunger.

18. A method of administering multiple-component formulations, the method comprises:
mixing multiple components using a mixing-system of claim 16 to form a first formulation, wherein the first chamber, the second chamber, and the third chamber comprise a first component, a second component, and a third component, respectively; and
administering the first formulation to a patient.

19. The method of claim 18, wherein the step of mixing the components comprises: mixing the first component with the third component, and mixing the mixture of the first component and the third component with the second component.

20. The method of claim 19, wherein the first component and the second component are in liquid form and the third component is in powder form.
